(19) United States
(12) Patent Application Publication
(10) Pub. No.: US 2023/0025119 A1
(43) Pub. Date: Jan. 26, 2023

(54) A METHOD OF MANUFACTURING AN OSTOMY APPLIANCE

(71) Applicant: Salts Healthcare Limited, Birmingham, West Midlands (GB)

(72) Inventor: Marcus ALLEN, Birmingham, West Midlands (GB)

(21) Appl. No.: 17/758,487

(22) PCT Filed: Jan. 6, 2021
(86) PCT No.: PCT/GB2021/050027

§ 371 Date: Jul. 7, 2022

(30) Foreign Application Priority Data
Jan. 7, 2020 (GB) 2000190.5

(51) Int. Cl. A61L 28/00; A61F 5/445

(52) U.S. Cl. CPC A61L 28/003; A61F 5/445

(57) ABSTRACT

A method of manufacturing an ostomy appliance including: a first wall and a second wall which are connected to each other at or near their peripheries to form a main body having an internal cavity for collecting waste, and an opening, and an outlet portion, formed from outlet layers and having an entrance, which is fluidly connected to the internal cavity, and an exit, which permits waste collected in the internal cavity to be emptied. The method can include attaching the outlet portion to the main body by: positioning the outlet portion, relative to the opening in the main body so that the entrance aligns with the opening, and positioning a soluble member such that the first wall and first outlet layer are positioned to one side of the soluble member and the second wall and second outlet layer positioned to an opposing side of the soluble member.

### TECHNICAL FIELD

The present invention relates to an ostomy appliance and a method of manufacturing an ostomy appliance.

### BACKGROUND AND SUMMARY

Ostomy appliances are well known medical devices that are manufactured in multiple different ways. The present invention relates to ostomy appliances that are manufactured from separate parts that are subsequently attached to each other.

The present invention aims to alleviate one or more problems with the prior art.

According to an aspect of the invention we provide a method of manufacturing an ostomy appliance, the ostomy appliance including: a first wall and a second wall which are connected to each other at or near their peripheries to form a main body, the main body having an internal cavity for collecting waste, and an opening, and an outlet portion, formed from first and second outlet layers and having an entrance, which is fluidly connected to the internal cavity, and an exit, which permits waste collected in the internal cavity to be emptied, the method of manufacturing the ostomy appliance including attaching the outlet portion to the main body by: positioning the outlet portion, relative to the opening in the main body so that the entrance aligns with the opening, and positioning a soluble member such that the first wall and first outlet layer are positioned to one side of the soluble member and the second wall and second outlet layer positioned to an opposing side of the soluble member, and attaching the first wall to the first outlet layer and attaching the second wall to the second outlet layer.

According to a second aspect of the invention we provide an ostomy appliance including: a first wall and a second wall which are connected to each other at or near their peripheries to form a main body, the main body having an internal cavity for collecting waste, an outlet portion including an entrance, which is fluidly connected to the internal cavity at an opening in the main body, and an exit, which permits waste collected in the internal cavity to be emptied, and wherein a soluble member is located inside the outlet portion and/or the internal cavity.

### BRIEF DESCRIPTION OF THE FIGURES

Some embodiments of the invention are described below with reference to FIGS. 1 to 4, of which:

FIG. 1 is a view of an ostomy appliance at a stage during manufacture,

FIG. 2 is a view of an ostomy appliance,

FIG. 3 is a side cross-sectional view of an ostomy appliance during manufacture, and

FIG. 4 is a side cross-sectional view of an ostomy appliance.

### DETAILED DESCRIPTION

Referring to the figures and specifically FIGS. 2 and 4, an ostomy appliance 100 is shown. The ostomy appliance 100 includes a main body 102 and an outlet portion 104.

The main body 102 is formed from a first wall 110 and a second wall 112, which are connected about their peripheries and provide an internal cavity 114 (which, in use, functions to collect waste). The first wall 110 has a stoma-receiving opening and a connection member 116 is attached to the first wall 110 aligning with the stoma-receiving opening. In the illustrated example, the connection member 116 is shown towards the top or upper part of the ostomy appliance 100. In use, the connection member 116 (which has an outward facing adhesive surface) adheres to the epidermis of the user about their stoma, so that the internal cavity 114 is in fluid connection with the stoma.

It should be appreciated that the connection member 116 is not essential to the present invention and may not be provided at all. In other words, a different mechanism of attaching the ostomy appliance 100 to the user may be provided (for example, the ostomy appliance 100 may be a “two-piece” arrangement where the main body 102 connects to another part that may already be attached to the user).

In some embodiments, the second wall 112 may include an air vent/opening (not shown) for air to escape the internal cavity 114 (which avoids the build-up of gas within the ostomy appliance 100). The air vent may include a filter across the opening to filter odours from the gas escaping the ostomy appliance 100.

In some embodiments, the main body 102 may include one, or more, further layers or walls (not shown) between the first and second walls 110, 112. These further layers may extend entirely or partially across the internal cavity 114 and provide an air pathway to the air vent/opening but prevents waste, which is entering the internal cavity, contacting the second wall 112 (thus, avoiding the first and second walls 110, 112 from sticking together and/or keeps the air vent free of waste material).

The outlet portion 104 is formed from a first outlet layer 130 and a second outlet layer 132 and has an entrance 104a and an exit 104b. The outlet portion 104 is connected to an opening in the main body 102, at its entrance 104a, and is fluidly connected to the internal cavity 114. The exit 104b of the outlet portion 104 permits waste collected in the internal cavity 114 to be emptied.

The purpose of the outlet portion 104 is to provide an exit of the ostomy appliance 100, so that the user can selectively empty their ostomy appliance 100 (for example, into a toilet or other receptacle) as and when they desire.

In some embodiments (such as the example illustrated in FIG. 2), the outlet portion 104 is foldable, so that the exit 104b folds upwards towards the main body 102. An attachment mechanism, for example hook and loop fasteners, are provided on two opposing portions of the outlet portion 104, so that the exit 104b of the outlet portion 104 can be retained in a folded configuration.

A soluble member 120 is located within the outlet portion 104 and/or the internal cavity 114. The use and positioning of the soluble member 120 is designed to facilitate an easier and more efficient manufacturing process (discussed below in more detail).

In some embodiments, the soluble member 120 is located in or adjacent the entrance 104a of the outlet portion 104. Furthermore, when the ostomy appliance 100 is fully formed, the soluble member 120 is also located in the opening in the main body 102. In some embodiments, the first wall 110 of the main body 102 is attached to the first outlet layer 130 at a first connection. The second wall 112 of the main body 102 is attached to the second outlet layer 132 at a second connection. The soluble member 120 is positioned between the first connection and the second connection.

In some embodiments, the soluble member 120 is positioned in appropriate location (i.e. where the connections are to be formed) before the first and second connections are formed.

In some embodiments, the soluble member 120 is water soluble (so that waste which is collected in the internal cavity 114 is able to dissolve the soluble member 120). In some embodiments, the soluble member 120 is made from one of paper (e.g. a cellulose based material), wafer paper, and rice paper. Although, it should be appreciated that any material that dissolves on contact with water and/or stoma waste may be suitable.

In some embodiments, the soluble member 120 may be made form a material which dissolves in the presence of another solvent (i.e. other than water). The soluble member 120 may be made from a material that is gas soluble and dissolves in air over a predetermined time that suits the period taken to manufacture the ostomy appliance 100.

It should be appreciated that the choice of material as the soluble member 120 is important, since an advantage is provided because there is no need to remove the layer at the end of the manufacturing process. The stages of manufacturing the ostomy appliance 100 will now be described in detail.

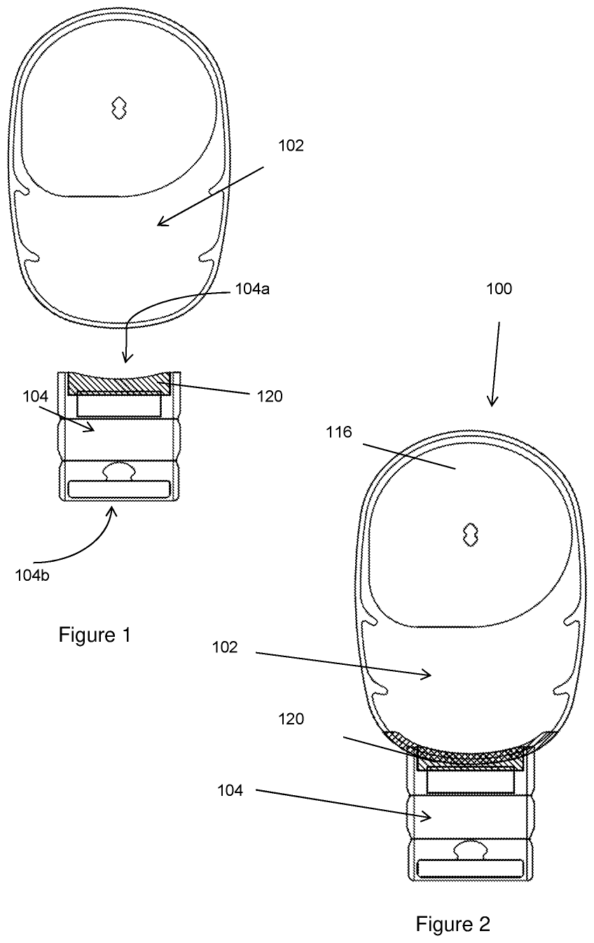

With reference in particular to FIGS. 1, 3 and 4, different stages of the manufacture of the ostomy appliance 100 are shown. As discussed above, the main body 102 is formed from the first and second walls 110, 112 and has the internal cavity 114 and the opening. The outlet portion 104 is formed from the first and second outlet layers 130, 132 and has the entrance 104a and the exit 104b.

The method of manufacturing the ostomy appliance 100 includes the steps of: attaching the outlet portion 104 to the main body 102 by positioning the outlet portion 104 relative to the opening in the main body 102 so that the entrance 104a aligns with the opening. The method includes positioning the soluble member 120 so that the first wall 110 and first outlet layer 130 are positioned to one side of the soluble member 120 and the second wall 112 and second outlet layer 132 are positioned to an opposing side of the soluble member 120. The purpose of the soluble member 120 is discussed in detail below, but generally functions to prevent first (i.e. the front) and second (i.e. the back) sides of the ostomy appliance 100 from becoming attached to each other during the manufacturing process.

The outlet portion 104 is then attached to the main body 102. Specifically, the method includes attaching the first wall 110 of the main body 102 to the first outlet layer 130 of the outlet portion 104 and attaching the second wall 112 to the second outlet layer 132.

In use, the outlet portion 104 provides a conduit out of the ostomy appliance 100. Thus, the exit 104b of the outlet portion 104 provides an exit from the internal cavity 114 in the main body 102.

In some embodiments, the method also includes the step of forming the main body 102 by attaching the first wall 110 and the second wall 112 together. The first and second walls 110, 112 are attached at or near their peripheries, so that the internal cavity 114 is provided in between. The step of attaching the first and second walls 110, 112 together also involves leaving an opening for receiving the outlet portion 104. In other words, a portion of the seam between the first and second wall 110, 112 is left open/unattached.

In embodiments, the first and second walls 110, 112 are welded together using a heat welding process. It should be appreciated that there are a number of processes that may provide the necessary sealing between the first and second walls 110, 112. For example, the method may use one of the following techniques: RF welding, impulse welding, ultrasonic welding and adhesive. A welding process may be advantageous as it requires less interaction with the area where the seams between the first and second walls 110, 112 are to be formed before the seam is formed and the main body cut out. This is because adhesive must be applied to one surface followed by positioning of the second surface to adhere too and then the main body would be cut out whereas a welding process only requires a single step for the seam formation.

In some embodiments, the step of attaching the first wall 110 and the second wall 112 together includes overlaying a first layer of material with a second layer of material. Subsequently, the shape of the main body 102 is formed by cutting out the desired shape and sealing the edges at the same time. In other words, the seams/sealing at the periphery of the first and second walls 110, 112 are formed at the same time as the walls are cut out from a larger piece of material. This may be advantageous because the connection between first and second walls 110, 112 is more reliable because there is no/reduced risk that the material will have moved prior to cutting the main body 102 out and, thus, the seals are in the correct location.

In some embodiments, a stoma-receiving opening is formed in the first wall 110. Multiple stoma-receiving openings may be cut from the first layer of material before multiple main bodies 102 are formed. For example, the stoma-receiving openings may be formed in a row at predetermined distances apart and then main bodies 102 may also be formed around those stoma-receiving openings at predetermined distances apart. In some embodiments, the adhesive members 116 are attached to the first layer of material before or as the stoma-receiving opening is formed. As such, multiple adhesive members 116 may be aligned and spaced and connected accordingly along the layer of material.

The formation of the outlet portion 104 and its attachment to the main body will now be described. Forming the outlet portion 104 involves connecting the first outlet layer 130 and the second outlet layer 132. Two elongate connection formations are formed by attaching the first outlet layer 130 to the second outlet layer 132, such that a conduit is formed between them. In some embodiments, the method includes positioning the soluble member 120 between the first outlet layer 130 and the second outlet layer 132 (and may be before the first outlet layer 130 and the second outlet layer 132 are attached together).

In this example, heat welding is used to form the outlet portion 104. It should be appreciated that the first outlet layer 130 may be attached to the second outlet layer 132 using another process, such as RF welding, impulse welding, ultrasonic welding and adhesive. For similar reasoning to that discussed above, a welding process may be favoured over an adhesive process.

The material to form the first outlet layer 130 may be taken from a sheet of material, which has more than one first outlet layers 130 positioned adjacent each other (or spaced a predetermined distance from each other). A second sheet of material may positioned on the first sheet and the connection formations to form the outlet portion 104 may be formed.

In some embodiments, excess material around the outlet portion 104 (i.e. material that is outside where the seams are formed between the two outlet layers 130, 132) is preferably trimmed/removed before the outlet portion 104 is moved into positioned relative to the main body 102, and may, in some embodiments, be trimmed/removed at the same time or in the same process as when the seams are formed.

During manufacture the soluble member 120 is located in the desired position, before the main body 102 can be attached to the outlet portion 104. The soluble member 120 provides a barrier between the first wall 110/first outlet layer 130 and the second wall 112/second outlet layer 132, so that when the outlet portion 104 is attached to the main body 102, the conduit from the internal cavity 114 to the exit is open (is not stuck together). Thus, the first wall 110 and the first outlet layer 130 are positioned on one side of the soluble member 120, and the second wall 112 and the second outlet layer 132 are on the opposing side of the soluble member 120. It is important that the soluble member 120 is positioned across the junction where the main body 102 and the outlet portion 104 overlap, so that the connection between the first wall 110 and the first outlet layer 130 and the connection between the second wall 112 and the second outlet later 132 are not connected together (remain independent connections e.g. separate seams).

In some embodiments, the soluble member 120 may be positioned in the opening of the main body 102 prior to the entrance 104a of the outlet portion 104 being positioned adjacent the opening, ready to be attached. In such an example, the soluble member 120 is positioned closer to the first and second outlet layers 130, 132 and further away from the first and second walls 110, 112. In other words, the first and second outlet layers 130, 132 of the outlet portion 104 are positioned inside the first and second walls 110, 112 of the main body 102 (i.e. the first outlet layer 130 is sandwiched between the soluble member 120 and the first wall 110 and the second outlet layer 132 is sandwiched between the soluble member 120 and the second wall 112).

Alternatively, the soluble member 120 is positioned within the entrance 104a of the outlet portion 104 (i.e. between the first outlet layer 130 and the second outlet layer 132) before the outlet portion 104 is positioned adjacent the opening in the main body 102 for attachment. In such an example, the soluble member 120 is positioned closer to the first and second wall 110, 112 and further away from the first and second outlet layers 130, 132. In other words, the first and second walls 110, 112 of the main body 102 are positioned inside the first and second outlet layers 130, 132 of the outlet portion 104 (i.e. the first wall 110 is sandwiched between the soluble member 120 and the first outlet layer 130 and the second wall 112 is sandwiched between the soluble member 120 and the second outlet layer 132).

In some embodiments, the entrance 104a of the outlet portion 104 is trimmed before it is moved into an “attachment position” relative to the main body 104. This is so that the soluble member 120 is positioned at the entrance 104a to the outlet portion 104 (so the placement of the soluble member 120 in between the first and second outlet layers 130, 132 can be adjusted by removing material at the entrance and, thus, the soluble member 120 is moved relative to the entrance 104a). This is advantageous because it is ensured then that the soluble member 120 is positioned in the correct position relative to the main body 102 (and more specifically, relative to the opening of the main body 102) once the outlet portion 104 is positioned for attachment.

The soluble member 120 may be positioned between the first outlet layer 130 and the second outlet layer 132 before the outlet layers 130, 132 are attached to each other to form the conduit.

In some embodiments, the soluble member is shaped and/or sized to correspond to the desired/manufactured shape of the outlet portion 104. In the present example, the soluble member 120 is generally rectangular in shape. The edges of the soluble member 120 correspond to the maximum width of the conduit formed in the outlet portion 104. In other words, the soluble member 120 extends across substantially the entire width of the (interior of the) outlet portion 104.

In the present example, the length of the soluble member 120 (i.e. the length that the soluble member 120 extends along the conduit of the outlet portion 104 away from the entrance 104a) is also chosen for advantageous reasons. In the illustrated example, the soluble member 120 is positioned at or next to the entrance 104a of the outlet portion and the length extends along the outlet portion 104. The length is chosen so that the soluble member extends across the entire area/seam that will be formed between the main body 102 and the outlet portion 104. In other words, the soluble member 120 extends across the area that forms the junction between the main body 102 and the outlet portion 104. This is illustrated particularly in FIG. 4 where welding elements 200 are shown at the junction between the main body 102 and the outlet portion 104 having formed the seam (and, thus, a seal) between the two. The soluble member 120 is shown at that junction.

Once the outlet portion 104 is formed (including positioning the soluble member 120 inside), the outlet portion 104 is positioned relative to the opening in the main body 102, so that the entrance 104a aligns with the opening.

Once the outlet portion 104 is positioned in the desired position relative to the main body 102, the outlet portion 104 is attached to the main body 102 in the opening. The parts of the first and second walls 110, 112 that were left open to form the opening are sealed against the respective layers of the outlet portion 104 to form a sealed main body 102. In other words, there is no exit for waste collected in the internal cavity 114 other than through the outlet portion 104.

In some embodiments, the first connection is formed by softening (i.e. using heat) one or both of the first wall 110 and the first outlet layer 130 until they fuse together. Likewise, the second connection is formed by softening one or both of the second wall 112 and the second outlet layer 132 until they fuse together.

In some embodiments, the process of attaching the first wall 110 to the first outlet layer 130 and the second wall 112 to the second outlet layer 132 is performed using a welding process (i.e. the first and second connections are formed using welding). The welding process may be one of heat welding, RF welding, impulse welding and ultrasonic welding. The soluble member 120 is positioned to stop the front/first side of the ostomy appliance 100 (i.e. the first wall 110 and first outlet layer 130) and the back/second side of the ostomy appliance 100 (i.e. the second wall 112 and second outlet layer 132) from melting into each other during the step of welding the outlet portion 104 to the main body 102.

In some embodiments, the first and second connections are formed synchronously.

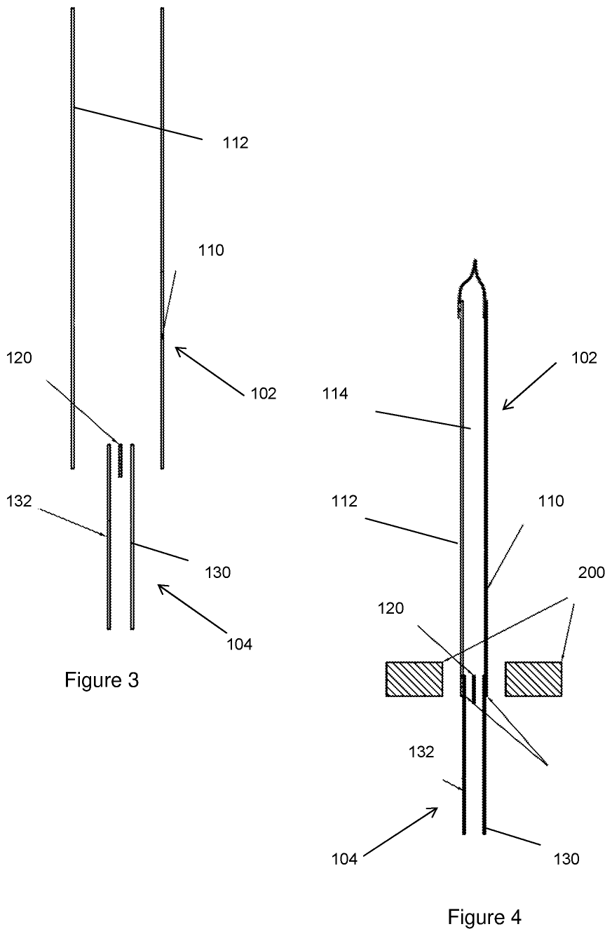

Different methods of attaching the outlet portion 104 to the main body 102 are outlined above—however, FIGS. 3 and 4 illustrate a cross section of the ostomy appliance 100 at different stages of manufacture using a heat welding process. FIG. 3 shows the outlet portion 104 positioned in the opening at the bottom of the main body 102. As can be seen in the figure, the soluble member 120 is positioned at the entrance 104a of the outlet portion 104 and at the base of the first and second walls 110, 112 of the main body 102 (i.e. at the opening), where the outlet portion 104 will be attached to the main body 102.

FIG. 4 illustrates an ostomy appliance 100 after the required seals have been made. Welding elements 200 are shown at the junction between the main body 102 and the outlet portion 104. The seals between the main body 102 and the outlet portion 104 have been formed and the soluble member 120 is positioned.

As mentioned above, the function of the soluble member 120 is to provide a barrier, between the layers 130, 132 of the outlet portion 104 when they are sealed to the main body 102, that does not melt (specifically does not melt into the material of the either side of the ostomy appliance 100). Thus, the conduit to the exit 104b from the internal cavity 114 remains open. In other words, the soluble member 120 prevents the walls of the main body 102 and the outlet portion 104 from sealing together. In this illustrated example, the first wall 110 and the first outlet layer 130 are sealed together (a first connection) and the second wall 112 and the second outlet layer 132 are sealed together (a second connection) and the soluble member 120 prevents a seal (or a permanent seal, at least) from forming between the first and second connection.

The exit 104b of the outlet portion 104 then becomes the outlet of the ostomy appliance 100. In use, the outlet portion 104 is selectively sealable (for example, the outlet portion 104 be foldable or have another mechanism to hold the exit 104b closed, as is known in the art). Thus, waste can be collected in the internal cavity 114 and then the user can choose to empty the appliance 100 as they wish by opening the exit 104b.

It should be appreciated that the steps discussed above may not occur specifically in the order outlined. For example, the outlet portion 104 may be formed on a separate manufacturing line/at a different time to the main body 104. Multiple outlet portions 104 may be formed and positioned in a predetermined pattern (for example, frangibly connected to each other on a roll that is ready to be unwound, disconnected and used). The same applies to the manufacture of the main body 102. The first and second wall 110, 112 may be formed into the main body 102 ready for the outlet portions 104 to be attached.

Once the essential steps of manufacturing have been completed, an ostomy appliance 100 has been created and the soluble member 120 remains in position. The above described method of manufacture is advantageous because the ostomy appliance 100 can be shipped to a user with the soluble member 120 still in place. There is no requirement to remove the soluble member 120 prior to use. The soluble member 120 is dissolvable in water and, as such, when it comes into contact with waste exiting a stoma, will dissolve. Therefore, once the ostomy appliance 100 is used, the soluble member 120 dissolves.

This provides an advantage over prior methods of manufacture that require more interaction to produce an ostomy appliance that can be shipped/is ready for use.

When used in this specification, the terms “comprises” and “comprising” and variations thereof mean that the specified features, steps or integers are included. The terms are not to be interpreted to exclude the presence of other features, steps or components.

The features disclosed in the foregoing description, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope is not intended to be limited solely to these embodiments.

### DESCRIPTION OF INVENTION

The present invention relates to an ostomy appliance and a method of manufacturing an ostomy appliance.

Ostomy appliances are well known medical devices that are manufactured in multiple different ways. The present invention relates to ostomy appliances that are manufactured from separate parts that are subsequently attached to each other.

The present invention aims to alleviate one or more problems with the prior art.

According to an aspect of the invention we provide a method of manufacturing an ostomy appliance, the ostomy appliance including: a first wall and a second wall which are connected to each other at or near their peripheries to form a main body, the main body having an internal cavity for collecting waste, and an opening, and an outlet portion, formed from first and second outlet layers and having an entrance, which is fluidly connected to the internal cavity, and an exit, which permits waste collected in the internal cavity to be emptied, the method of manufacturing the ostomy appliance including attaching the outlet portion to the main body by: positioning the outlet portion, relative to the opening in the main body so that the entrance aligns with the opening, and positioning a soluble member such that the first wall and first outlet layer are positioned to one side of the soluble member and the second wall and second outlet layer positioned to an opposing side of the soluble member, and attaching the first wall to the first outlet layer and attaching the second wall to the second outlet layer.

According to a second aspect of the invention we provide an ostomy appliance including: a first wall and a second wall which are connected to each other at or near their peripheries to form a main body, the main body having an internal cavity for collecting waste, an outlet portion including an entrance, which is fluidly connected to the internal cavity at an opening in the main body, and an exit, which permits waste collected in the internal cavity to be emptied, and wherein a soluble member is located inside the outlet portion and/or the internal cavity.

Further features of different aspects of the invention are recited in the appended claims.

Some embodiments of the invention are described below with reference to FIGS. 1 to 4, of which:

FIG. 1 is a view of an ostomy appliance at a stage during manufacture,

FIG. 2 is a view of an ostomy appliance,

FIG. 3 is a side cross-sectional view of an ostomy appliance during manufacture, and

FIG. 4 is a side cross-sectional view of an ostomy appliance.

Referring to the figures and specifically FIGS. 2 and 4, an ostomy appliance 100 is shown. The ostomy appliance 100 includes a main body 102 and an outlet portion 104.

The main body 102 is formed from a first wall 110 and a second wall 112, which are connected about their peripheries and provide an internal cavity 114 (which, in use, functions to collect waste). The first wall 110 has a stoma-receiving opening and a connection member 116 is attached to the first wall 110 aligning with the stoma-receiving opening. In the illustrated example, the connection member 116 is shown towards the top or upper part of the ostomy appliance 100. In use, the connection member 116 (which has an outward facing adhesive surface) adheres to the epidermis of the user about their stoma, so that the internal cavity 114 is in fluid connection with the stoma.

It should be appreciated that the connection member 116 is not essential to the present invention and may not be provided at all. In other words, a different mechanism of attaching the ostomy appliance 100 to the user may be provided (for example, the ostomy appliance 100 may be a “two-piece” arrangement where the main body 102 connects to another part that may already be attached to the user).

In some embodiments, the second wall 112 may include an air vent/opening (not shown) for air to escape the internal cavity 114 (which avoids the build-up of gas within the ostomy appliance 100). The air vent may include a filter across the opening to filter odours from the gas escaping the ostomy appliance 100.

In some embodiments, the main body 102 may include one, or more, further layers or walls (not shown) between the first and second walls 110, 112. These further layers may extend entirely or partially across the internal cavity 114 and provide an air pathway to the air vent/opening but prevents waste, which is entering the internal cavity, contacting the second wall 112 (thus, avoiding the first and second walls 110, 112 from sticking together and/or keeps the air vent free of waste material).

The outlet portion 104 is formed from a first outlet layer 130 and a second outlet layer 132 and has an entrance 104a and an exit 104b. The outlet portion 104 is connected to an opening in the main body 102, at its entrance 104a, and is fluidly connected to the internal cavity 114. The exit 104b of the outlet portion 104 permits waste collected in the internal cavity 114 to be emptied.

The purpose of the outlet portion 104 is to provide an exit of the ostomy appliance 100, so that the user can selectively empty their ostomy appliance 100 (for example, into a toilet or other receptacle) as and when they desire.

In some embodiments (such as the example illustrated in FIG. 2), the outlet portion 104 is foldable, so that the exit 104b folds upwards towards the main body 102. An attachment mechanism, for example hook and loop fasteners, are provided on two opposing portions of the outlet portion 104, so that the exit 104b of the outlet portion 104 can be retained in a folded configuration.

A soluble member 120 is located within the outlet portion 104 and/or the internal cavity 114. The use and positioning of the soluble member 120 is designed to facilitate an easier and more efficient manufacturing process (discussed below in more detail).

In some embodiments, the soluble member 120 is located in or adjacent the entrance 104a of the outlet portion 104. Furthermore, when the ostomy appliance 100 is fully formed, the soluble member 120 is also located in the opening in the main body 102. In some embodiments, the first wall 110 of the main body 102 is attached to the first outlet layer 130 at a first connection. The second wall 112 of the main body 102 is attached to the second outlet layer 132 at a second connection. The soluble member 120 is positioned between the first connection and the second connection.

In some embodiments, the soluble member 120 is positioned in appropriate location (i.e. where the connections are to be formed) before the first and second connections are formed.

In some embodiments, the soluble member 120 is water soluble (so that waste which is collected in the internal cavity 114 is able to dissolve the soluble member 120). In some embodiments, the soluble member 120 is made from one of paper (e.g. a cellulose based material), wafer paper, and rice paper. Although, it should be appreciated that any material that dissolves on contact with water and/or stoma waste may be suitable.

In some embodiments, the soluble member 120 may be made form a material which dissolves in the presence of another solvent (i.e. other than water). The soluble member 120 may be made from a material that is gas soluble and dissolves in air over a predetermined time that suits the period taken to manufacture the ostomy appliance 100.

It should be appreciated that the choice of material as the soluble member 120 is important, since an advantage is provided because there is no need to remove the layer at the end of the manufacturing process. The stages of manufacturing the ostomy appliance 100 will now be described in detail.

With reference in particular to FIGS. 1, 3 and 4, different stages of the manufacture of the ostomy appliance 100 are shown. As discussed above, the main body 102 is formed from the first and second walls 110, 112 and has the internal cavity 114 and the opening. The outlet portion 104 is formed from the first and second outlet layers 130, 132 and has the entrance 104a and the exit 104b.

The method of manufacturing the ostomy appliance 100 includes the steps of: attaching the outlet portion 104 to the main body 102 by positioning the outlet portion 104 relative to the opening in the main body 102 so that the entrance 104a aligns with the opening. The method includes positioning the soluble member 120 so that the first wall 110 and first outlet layer 130 are positioned to one side of the soluble member 120 and the second wall 112 and second outlet layer 132 are positioned to an opposing side of the soluble member 120. The purpose of the soluble member 120 is discussed in detail below, but generally functions to prevent first (i.e. the front) and second (i.e. the back) sides of the ostomy appliance 100 from becoming attached to each other during the manufacturing process.

The outlet portion 104 is then attached to the main body 102. Specifically, the method includes attaching the first wall 110 of the main body 102 to the first outlet layer 130 of the outlet portion 104 and attaching the second wall 112 to the second outlet layer 132.

In use, the outlet portion 104 provides a conduit out of the ostomy appliance 100. Thus, the exit 104b of the outlet portion 104 provides an exit from the internal cavity 114 in the main body 102.

In some embodiments, the method also includes the step of forming the main body 102 by attaching the first wall 110 and the second wall 112 together. The first and second walls 110, 112 are attached at or near their peripheries, so that the internal cavity 114 is provided in between. The step of attaching the first and second walls 110, 112 together also involves leaving an opening for receiving the outlet portion 104. In other words, a portion of the seam between the first and second wall 110, 112 is left open/unattached.

In embodiments, the first and second walls 110, 112 are welded together using a heat welding process. It should be appreciated that there are a number of processes that may provide the necessary sealing between the first and second walls 110, 112. For example, the method may use one of the following techniques: RF welding, impulse welding, ultrasonic welding and adhesive. A welding process may be advantageous as it requires less interaction with the area where the seams between the first and second walls 110, 112 are to be formed before the seam is formed and the main body cut out. This is because adhesive must be applied to one surface followed by positioning of the second surface to adhere too and then the main body would be cut out whereas a welding process only requires a single step for the seam formation.

In some embodiments, the step of attaching the first wall 110 and the second wall 112 together includes overlaying a first layer of material with a second layer of material. Subsequently, the shape of the main body 102 is formed by cutting out the desired shape and sealing the edges at the same time. In other words, the seams/sealing at the periphery of the first and second walls 110, 112 are formed at the same time as the walls are cut out from a larger piece of material. This may be advantageous because the connection between first and second walls 110, 112 is more reliable because there is no/reduced risk that the material will have moved prior to cutting the main body 102 out and, thus, the seals are in the correct location.

In some embodiments, a stoma-receiving opening is formed in the first wall 110. Multiple stoma-receiving openings may be cut from the first layer of material before multiple main bodies 102 are formed. For example, the stoma-receiving openings may be formed in a row at predetermined distances apart and then main bodies 102 may also be formed around those stoma-receiving openings at predetermined distances apart. In some embodiments, the adhesive members 116 are attached to the first layer of material before or as the stoma-receiving opening is formed. As such, multiple adhesive members 116 may be aligned and spaced and connected accordingly along the layer of material.

The formation of the outlet portion 104 and its attachment to the main body will now be described. Forming the outlet portion 104 involves connecting the first outlet layer 130 and the second outlet layer 132. Two elongate connection formations are formed by attaching the first outlet layer 130 to the second outlet layer 132, such that a conduit is formed between them. In some embodiments, the method includes positioning the soluble member 120 between the first outlet layer 130 and the second outlet layer 132 (and may be before the first outlet layer 130 and the second outlet layer 132 are attached together).

In this example, heat welding is used to form the outlet portion 104. It should be appreciated that the first outlet layer 130 may be attached to the second outlet layer 132 using another process, such as RF welding, impulse welding, ultrasonic welding and adhesive. For similar reasoning to that discussed above, a welding process may be favoured over an adhesive process.

The material to form the first outlet layer 130 may be taken from a sheet of material, which has more than one first outlet layers 130 positioned adjacent each other (or spaced a predetermined distance from each other). A second sheet of material may positioned on the first sheet and the connection formations to form the outlet portion 104 may be formed.

In some embodiments, excess material around the outlet portion 104 (i.e. material that is outside where the seams are formed between the two outlet layers 130, 132) is preferably trimmed/removed before the outlet portion 104 is moved into positioned relative to the main body 102, and may, in some embodiments, be trimmed/removed at the same time or in the same process as when the seams are formed.

During manufacture the soluble member 120 is located in the desired position, before the main body 102 can be attached to the outlet portion 104. The soluble member 120 provides a barrier between the first wall 110/first outlet layer 130 and the second wall 112/second outlet layer 132, so that when the outlet portion 104 is attached to the main body 102, the conduit from the internal cavity 114 to the exit is open (is not stuck together). Thus, the first wall 110 and the first outlet layer 130 are positioned on one side of the soluble member 120, and the second wall 112 and the second outlet layer 132 are on the opposing side of the soluble member 120. It is important that the soluble member 120 is positioned across the junction where the main body 102 and the outlet portion 104 overlap, so that the connection between the first wall 110 and the first outlet layer 130 and the connection between the second wall 112 and the second outlet later 132 are not connected together (remain independent connections e.g. separate seams).

In some embodiments, the soluble member 120 may be positioned in the opening of the main body 102 prior to the entrance 104a of the outlet portion 104 being positioned adjacent the opening, ready to be attached. In such an example, the soluble member 120 is positioned closer to the first and second outlet layers 130, 132 and further away from the first and second walls 110, 112. In other words, the first and second outlet layers 130, 132 of the outlet portion 104 are positioned inside the first and second walls 110, 112 of the main body 102 (i.e. the first outlet layer 130 is sandwiched between the soluble member 120 and the first wall 110 and the second outlet layer 132 is sandwiched between the soluble member 120 and the second wall 112).

Alternatively, the soluble member 120 is positioned within the entrance 104a of the outlet portion 104 (i.e. between the first outlet layer 130 and the second outlet layer 132) before the outlet portion 104 is positioned adjacent the opening in the main body 102 for attachment. In such an example, the soluble member 120 is positioned closer to the first and second wall 110, 112 and further away from the first and second outlet layers 130, 132. In other words, the first and second walls 110, 112 of the main body 102 are positioned inside the first and second outlet layers 130, 132 of the outlet portion 104 (i.e. the first wall 110 is sandwiched between the soluble member 120 and the first outlet layer 130 and the second wall 112 is sandwiched between the soluble member 120 and the second outlet layer 132).

In some embodiments, the entrance 104a of the outlet portion 104 is trimmed before it is moved into an “attachment position” relative to the main body 104. This is so that the soluble member 120 is positioned at the entrance 104a to the outlet portion 104 (so the placement of the soluble member 120 in between the first and second outlet layers 130, 132 can be adjusted by removing material at the entrance and, thus, the soluble member 120 is moved relative to the entrance 104a). This is advantageous because it is ensured then that the soluble member 120 is positioned in the correct position relative to the main body 102 (and more specifically, relative to the opening of the main body 102) once the outlet portion 104 is positioned for attachment.

The soluble member 120 may be positioned between the first outlet layer 130 and the second outlet layer 132 before the outlet layers 130, 132 are attached to each other to form the conduit.

In some embodiments, the soluble member is shaped and/or sized to correspond to the desired/manufactured shape of the outlet portion 104. In the present example, the soluble member 120 is generally rectangular in shape. The edges of the soluble member 120 correspond to the maximum width of the conduit formed in the outlet portion 104. In other words, the soluble member 120 extends across substantially the entire width of the (interior of the) outlet portion 104.

In the present example, the length of the soluble member 120 (i.e. the length that the soluble member 120 extends along the conduit of the outlet portion 104 away from the entrance 104a) is also chosen for advantageous reasons. In the illustrated example, the soluble member 120 is positioned at or next to the entrance 104a of the outlet portion and the length extends along the outlet portion 104. The length is chosen so that the soluble member extends across the entire area/seam that will be formed between the main body 102 and the outlet portion 104. In other words, the soluble member 120 extends across the area that forms the junction between the main body 102 and the outlet portion 104. This is illustrated particularly in FIG. 4 where welding elements 200 are shown at the junction between the main body 102 and the outlet portion 104 having formed the seam (and, thus, a seal) between the two. The soluble member 120 is shown at that junction.

Once the outlet portion 104 is formed (including positioning the soluble member 120 inside), the outlet portion 104 is positioned relative to the opening in the main body 102, so that the entrance 104a aligns with the opening.

Once the outlet portion 104 is positioned in the desired position relative to the main body 102, the outlet portion 104 is attached to the main body 102 in the opening. The parts of the first and second walls 110, 112 that were left open to form the opening are sealed against the respective layers of the outlet portion 104 to form a sealed main body 102. In other words, there is no exit for waste collected in the internal cavity 114 other than through the outlet portion 104.

In some embodiments, the first connection is formed by softening (i.e. using heat) one or both of the first wall 110 and the first outlet layer 130 until they fuse together. Likewise, the second connection is formed by softening one or both of the second wall 112 and the second outlet layer 132 until they fuse together.

In some embodiments, the process of attaching the first wall 110 to the first outlet layer 130 and the second wall 112 to the second outlet layer 132 is performed using a welding process (i.e. the first and second connections are formed using welding). The welding process may be one of heat welding, RF welding, impulse welding and ultrasonic welding. The soluble member 120 is positioned to stop the front/first side of the ostomy appliance 100 (i.e. the first wall 110 and first outlet layer 130) and the back/second side of the ostomy appliance 100 (i.e. the second wall 112 and second outlet layer 132) from melting into each other during the step of welding the outlet portion 104 to the main body 102.

In some embodiments, the first and second connections are formed synchronously.

Different methods of attaching the outlet portion 104 to the main body 102 are outlined above—however, FIGS. 3 and 4 illustrate a cross section of the ostomy appliance 100 at different stages of manufacture using a heat welding process. FIG. 3 shows the outlet portion 104 positioned in the opening at the bottom of the main body 102. As can be seen in the figure, the soluble member 120 is positioned at the entrance 104a of the outlet portion 104 and at the base of the first and second walls 110, 112 of the main body 102 (i.e. at the opening), where the outlet portion 104 will be attached to the main body 102.

FIG. 4 illustrates an ostomy appliance 100 after the required seals have been made. Welding elements 200 are shown at the junction between the main body 102 and the outlet portion 104. The seals between the main body 102 and the outlet portion 104 have been formed and the soluble member 120 is positioned.

As mentioned above, the function of the soluble member 120 is to provide a barrier, between the layers 130, 132 of the outlet portion 104 when they are sealed to the main body 102, that does not melt (specifically does not melt into the material of the either side of the ostomy appliance 100). Thus, the conduit to the exit 104b from the internal cavity 114 remains open. In other words, the soluble member 120 prevents the walls of the main body 102 and the outlet portion 104 from sealing together. In this illustrated example, the first wall 110 and the first outlet layer 130 are sealed together (a first connection) and the second wall 112 and the second outlet layer 132 are sealed together (a second connection) and the soluble member 120 prevents a seal (or a permanent seal, at least) from forming between the first and second connection.

The exit 104b of the outlet portion 104 then becomes the outlet of the ostomy appliance 100. In use, the outlet portion 104 is selectively sealable (for example, the outlet portion 104 be foldable or have another mechanism to hold the exit 104b closed, as is known in the art). Thus, waste can be collected in the internal cavity 114 and then the user can choose to empty the appliance 100 as they wish by opening the exit 104b.

It should be appreciated that the steps discussed above may not occur specifically in the order outlined. For example, the outlet portion 104 may be formed on a separate manufacturing line/at a different time to the main body 104. Multiple outlet portions 104 may be formed and positioned in a predetermined pattern (for example, frangibly connected to each other on a roll that is ready to be unwound, disconnected and used). The same applies to the manufacture of the main body 102. The first and second wall 110, 112 may be formed into the main body 102 ready for the outlet portions 104 to be attached.

Once the essential steps of manufacturing have been completed, an ostomy appliance 100 has been created and the soluble member 120 remains in position. The above described method of manufacture is advantageous because the ostomy appliance 100 can be shipped to a user with the soluble member 120 still in place. There is no requirement to remove the soluble member 120 prior to use. The soluble member 120 is dissolvable in water and, as such, when it comes into contact with waste exiting a stoma, will dissolve. Therefore, once the ostomy appliance 100 is used, the soluble member 120 dissolves.

This provides an advantage over prior methods of manufacture that require more interaction to produce an ostomy appliance that can be shipped/is ready for use.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. A method of manufacturing an ostomy appliance, the ostomy appliance including:
a first wall and a second wall which are connected to each other at or near their peripheries to form a main body,
the main body having an internal cavity for collecting waste, and an opening, and
an outlet portion, formed from first and second outlet layers and having an entrance, which is fluidly connected to the internal cavity, and an exit, which permits waste collected in the internal cavity to be emptied,
the method of manufacturing the ostomy appliance including attaching the outlet portion to the main body by:
positioning the outlet portion, relative to the opening in the main body so that the entrance aligns with the opening, and positioning a soluble member such that the first wall and first outlet layer are positioned to one side of the soluble member and the second wall and second outlet layer positioned to an opposing side of the soluble member, and
attaching the first wall to the first outlet layer and attaching the second wall to the second outlet layer.

2. The method according to claim 1, wherein the soluble member is positioned between the first and second outlet layers, prior to the outlet portion being positioned at the opening of the main body.

3. The method according to claim 2, wherein the first outlet layer is positioned closer to the soluble member than the first wall and the second outlet layer is positioned closer to the soluble member than the second wall.

4. The method according to claim 1, wherein the soluble member is positioned between the first wall and the second wall at the opening of the main body before the outlet portion is portioned for attachment to the main body.

5. The method of manufacturing an ostomy appliance according to claim 4, wherein the first wall is positioned closer to the soluble member than the first outlet layer and the second wall is positioned closer to the soluble member than the second outlet layer.

6. The method of manufacturing an ostomy appliance according to claim 1, wherein the method includes forming the outlet portion by attaching the first outlet layer to the second outlet layer.

7. The method of manufacturing an ostomy appliance according to claim 6, wherein the first outlet layer is attached to the second outlet layer along two elongate connection formations, such that a conduit is formed between the entrance and the exit.

8. The method of manufacturing an ostomy appliance according to claim 6, wherein the soluble member is positioned between the first outlet layer and the second outlet layer prior to the first and second outlet layers being attached to each other.

9. The method of manufacturing an ostomy appliance according to claim 6, wherein the first outlet layer is attached to the second outlet layer using at least one of heat welding, RF welding, impulse welding, ultrasonic welding and adhesive.

10. The method of manufacturing an ostomy appliance according to claim 6, wherein material from the first and second outlet layers is removed from the entrance, before the outlet portion is positioned relative to the opening of the main body.

11. The method of manufacturing an ostomy appliance according to claim 10, wherein the material removed adjusts a position of the entrance of the outlet portion.

12. The method of manufacturing an ostomy appliance according to claim 1, wherein the soluble member is shaped/sized to fit inside the outlet portion.

13. The method of manufacturing an ostomy appliance according to claim 1, wherein the first wall and the first outlet layer are attached to each other at a first connection and the second wall and the second outlet layer are attached to each other at a second connection.

14. The method of manufacturing an ostomy appliance according to claim 13, wherein the soluble member is positioned such that the first and second connections are formed either side of the soluble member.

15. The method of manufacturing an ostomy appliance according to claim 13, wherein the first connection is formed by softening one or both of the first wall and the first outlet layer until they fuse together and wherein the second connection is formed by softening one or both of the second wall and the second outlet layer until they fuse together.

16. The method according to claim 13, wherein the first and second connections are formed synchronously.

17. The method of manufacturing an ostomy appliance according to claim 1, wherein attaching the first wall to the first outlet layer and attaching the second wall to the second outlet layer is achieved using at least one of heat welding, RF welding, impulse welding and ultrasonic welding.

18. The method of manufacturing an ostomy appliance according to claim 1, wherein the method includes forming the main body by attaching the first wall and the second wall together at or near their peripheries'.

19. The method of manufacturing an ostomy appliance according to claim 18, wherein attaching the first wall and the second wall together includes overlaying a first layer of material with a second layer of material and attaching the first and second layers together and cutting the main body from the material at substantially the same time.

20. The method of manufacturing an ostomy appliance according to claim 18, wherein attaching the first wall and the second wall together is achieved using at least one of heat welding, RF welding, impulse welding, ultrasonic welding and adhesive.

21. The method of manufacturing an ostomy appliance according to claim 1 which further includes forming a stoma-receiving opening in the first wall.

22. An ostomy appliance including:
a first wall and a second wall which are connected to each other at or near their peripheries to form a main body,
the main body having an internal cavity for collecting waste,
an outlet portion including an entrance, which is fluidly connected to the internal cavity at an opening in the main body, and an exit, which permits waste collected in the internal cavity to be emptied, and wherein a soluble member is located inside the outlet portion and/or the internal cavity.

23. The ostomy appliance according to claim 22, wherein the outlet portion includes a first outlet layer and a second outlet layer which are connected to provide a conduit between the entrance and exit, and the first wall of the main body is attached to the first outlet layer at a first connection and the second wall of the main body is attached to the second outlet layer at a second connection and the soluble member is positioned between the first connection and the second connection.

24. The ostomy appliance according to claim 22, wherein the soluble member is located in or adjacent the entrance of the outlet portion, and/or wherein the soluble member is dissolvable by waste which is collected in the internal cavity.

25. The ostomy appliance according to claim 22, wherein the soluble member is made from a water soluble material and optionally the water soluble material comprises one of water dissolvable paper, wafer paper and rice paper.
